(19) United States
(12) Patent Application Publication
(10) Pub. No.: US 2024/0315741 A1
(43) Pub. Date: Sep. 26, 2024

(54) A METHOD AND SYSTEM FOR VERIFYING A CORRECTION OF A SPINAL CURVATURE BY IMAGING AND TRACKING

(71) Applicant: NEO MEDICAL SA, Villette (CH)

(72) Inventor: Vincent LEFAUCONNIER, Brent (CH)

(21) Appl. No.: 18/578,139

(22) PCT Filed: Jun. 22, 2022
(86) PCT No.: PCT/IB2022/055780

§ 371 Date: Jan. 10, 2024

(30) Foreign Application Priority Data
Jul. 13, 2021 (WO) PCT/IB2021/056309

(51) Int. Cl. A61B 17/70; A61B 34/10; A61B 34/20; A61B 90/00

(52) U.S. Cl. CPC A61B 17/7085; A61B 90/39; A61B 2034/104; A61B 2034/2057

(57) ABSTRACT

A method for determining a spinal rod for correcting a curvature of a spinal column of a living being, including the steps of detecting a rod attachment position for each pedicle screw by capturing image data from the pedicle screws at a surgical incision determining first parameters of the uncorrected spinal column with a data processing device entering second parameters of a desired arrangement of a desired corrected spinal column and calculating data characterizing a corrective spinal rod for achieving the desired corrected spinal column when the corrective spinal rod is attached to the pedicle screws, the data calculated based on the rod attachment positions and the second parameters.

### CROSS-REFERENCE TO RELATED APPLICATIONS

The present invention claims priority to International Patent Application No. PCT/IB2021/056309 that was filed on Jul. 13, 2021, the entire contents of this reference herewith incorporated by reference in its entirety.

The present invention is also related to and fully incorporates by reference International Patent Application No. PCT/IB2021/051694 that was filed on Mar. 1, 2021, International Patent Application No. PCT/IB2021/056242 that was filed on Jul. 12, 2021, and International Patent Application No. PCT/IB2022/051805 that was filed on Mar. 1, 2022.

### FIELD OF THE INVENTION

The present invention relates to the field of orthopedic surgery using image data processing to aid a surgeon or operator to perform the surgery, for example to determine positions of the vertebrae of the spinal column. In addition, the present invention related to a method, system, and device for using image data processing to provide for assistance or facilitation to a surgeon performing orthopedic surgery to verify whether a curvature of a spinal column has been corrected sufficiently.

### BACKGROUND

In the field of orthopedics and implant tools and systems for orthopedic surgery, more specifically spinal fusion surgery for a spinal column, and for correcting a curvature of a spinal column, a plurality of pedicle screws can be used to be attach to different vertebra with a bone anchor, through an incision location in the skin of the back of the patient. After several pedicle screws are attached to different vertebrae, the heads of these pedicle screws can be connected together with a rod-type or bar-type device, and the rod-type or bar-type device, also called spinal rod, is attached to the head of the pedicle screws with a set screw. As an example, for several adjacent vertebrae for vertebrae fusion, for each vertebra, usually two pedicle screws are screwably attached thereto with the bone anchor of the pedicle screw, and thereafter, these pedicle screws are mechanically fastened relative to each other by the use of the spinal rod that is placed in a groove or U-shaped opening of the pedicle screw head, forming a row of connected pedicle screws along the spinal column. This allows to provide for the mechanical support needed for spinal stabilization for spinal fusion in a patient or living being, and also to depart a specific curvature to the spinal column for correcting spinal deformations.

However, for correcting a curvature of the spinal column, orthopedic surgeons still rely on a rather informal approach for spinal correction and stabilization, by determining a shape and curvature of the corrective spinal rod based on experience, without any support in determining a rod shape, curvature and length. Therefore, in light of these deficiencies of the background art, strongly improved and novel methods for determining a spinal rod, and for analyzing and comparing the corrected spinal column post-correction are strongly desired.

### SUMMARY

According to one aspect of the present invention, a method for determining a spinal rod for correcting a curvature of a spinal column of a living being is provided. Preferably, the method includes the steps of detecting a rod attachment position for each pedicle screw by capturing image data from the pedicle screws at a surgical incision. determining first parameters of the uncorrected spinal column with a data processing device, entering second parameters of a desired arrangement of a desired corrected spinal column, and calculating data characterizing a corrective spinal rod for achieving the desired corrected spinal column when the corrective spinal rod is attached to the pedicle screws, the data calculated based on the rod attachment positions and the second parameters.

In a variant, the method preferably further includes a step of performing medical imaging to capture medical imaging data of the uncorrected spinal column, wherein the step of determining the first parameters can calculate the first parameters based on the captured medical imaging data. In another variant, the step of determining the first parameters can preferably calculate the first parameters based on the rod attachment positions of the pedicle screws of the step of detecting, or based data from a step of detecting a position of the pedicle screws by capturing image data from the pedicle screws.

Moreover, according to another aspect of the present invention, a non-transitory computer-readable medium is provided, the non-transitory computer-readable medium having computer instructions recorded thereon, the computer instructions configured to perform the different steps of the method for determining a spinal rod for correcting a curvature of a spinal column of a living being, when the computer instructions are executed on a data processing device.

In addition, according to still another aspect of the present invention, a system is provided, the system including a data processing device and at least one camera that is operatively connected to the data processing device, the data processing device configured to perform the steps of the method for determining a spinal rod for correcting a curvature of a spinal column of a living being.

The above and other objects, features and advantages of the present invention and the manner of realizing them will become more apparent, and the invention itself will best be understood from a study of the following description and appended claims with reference to the attached drawings showing some preferred embodiments of the invention.

### BRIEF DESCRIPTION OF THE SEVERAL DRAWINGS

The accompanying drawings, which are incorporated herein and constitute part of this specification, illustrate the presently preferred embodiments of the invention, and together with the general description given above and the detailed description given below, serve to explain features of the invention.

FIG. 1A shows a schematic overview of the steps of the method 300 for determining spinal correction rods R and for verifying a correction imparted to a spinal column SC, with an exemplary flowchart, according to an aspect of the present invention:

FIG. 1B shows a perspective and simplified view of a system 400 for performing the method 300, for example to perform one or more steps of medical imaging, steps of orthopedic surgery, and steps of scanning of the surgical location with a portable data processing device 100, the system 400 including a medical imaging device 310, a data processing device 320, and a portable data processing device 100 that can be operated by operator or surgeon O, according to another aspect of the present invention:

FIG. 1C shows a simplified and schematic back view of seven (7) exemplary vertebrae of a spinal column SC before and after corrective back surgery, with a correction of the Cobb's angle β to become smaller, after surgery:

FIGS. 2A-2E show simplified and schematic back or frontal views of a spinal column SC showing an exemplary number of seven (7) vertebrae V1 to V7, and rods R1, R2 for the spinal column correction, with FIG. 2A showing an exemplary section of a spinal column SC1 pre-surgery having an unhealthy large Cobb angle β, the representation resulting from data of medical imaging, FIG. 2B showing a representation of the location of pairs of attachment points AP of the different vertebrae V of a non-corrected spinal columns SC1, resulting from a scanning step M40 of method 300, FIG. 2C showing a desired, aspirational spinal column SC2 that is based on spinal parametrization values PAR2 that the surgeon, user, or operator O wants to achieve, for example with the Cobb angle β being zero, FIG. 2D showing a pair of exemplary spine correction rods R1, R2, that have been determined to bring spinal column SC to a new corrected position as shown in FIG. 2C, based on user—or computer-defined parametrization values PAR2, and FIG. 2E showing an exemplary view of a graphical user interface GUI displaying a rod template RT at the scale of 1:1 on a display device 120, 330;

FIG. 3 shows an exemplary and simplified graphical representation of the actual spinal column SC1 and the desired spinal column SC2 after surgical correction, for example as displayed on display device 120, 330 by a step M50, allowing the user, operator, or surgeon O to enter data that characterizes the desired spinal column SC2, and allows a graphical representation of the desired spinal column SC2; and

FIGS. 4A and 4B show a section of the spinal column SC with an exemplary number of three (3) vertebra for illustration purposes, with FIG. 4A showing an uncorrected, bent, original spinal column SC1 having an unhealthy Cobb angle β, and with FIG. 4B showing a desired, corrected spinal column SC2 with the Cobb angle being zero.

Herein, identical reference numerals are used, where possible, to designate identical elements that are common to the figures. Also, the images in the drawings are simplified for illustration purposes and may not be depicted to scale.

### DETAILED DESCRIPTION OF THE EMBODIMENTS

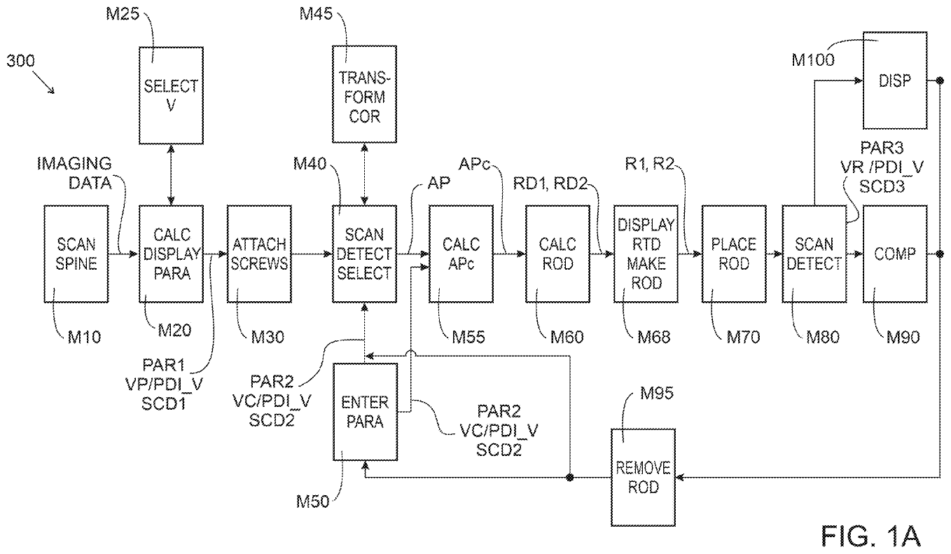

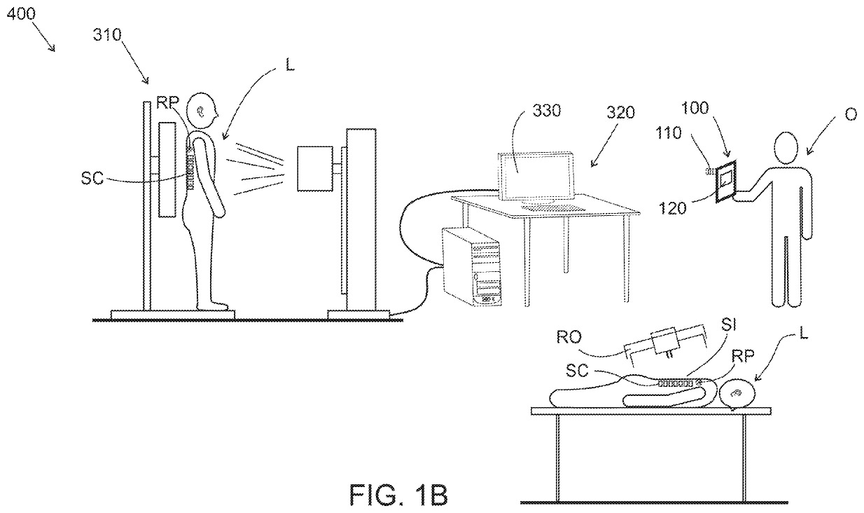

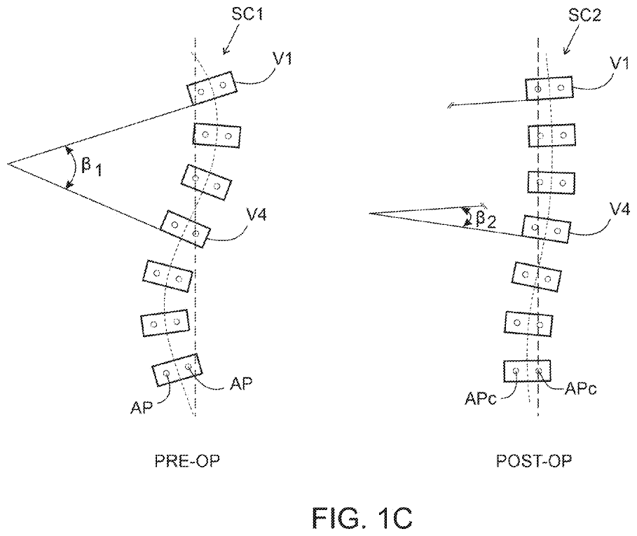

FIG. 1A shows a schematic view of the different steps that can be performed by method 300, method 300 including steps to assist a spinal correction surgery that is performed by a user, surgeon, or operator O, according to one aspect of the present invention. Method 300 can be used to determine data that characterizes a spinal correction rod R to depart a spinal correction to a spinal column SC, and can also be used to verify an impact of the proposed spinal correction rod R that can be attached to a series of pedicle screws PS of uncorrected spinal column SC1, for example but not limited to spinal rod data RD1, RD2 for a pair of rods R1, R2 that can be attached in parallel to each other to spinal column SC1. FIG. 1B shows a view of an exemplary system 400 that can be used to perform method 300, system 400 preferably including a medical imaging device 310, a data processing device 320 that is in operative connection with medical imaging system 310 to receive medical imaging data, and a portable data processing device 100 that includes a camera 110 or other image capturing device, and a display screen 120. The different data processing steps of method 300 can be performed at the medical imaging system 310, at the data processing device 320, or at the portable data processing device 100, or by another remote data processing device that is accessible through a network, for example a cloud-based data processing device such as a server, or by a one or a combination of the different data processing devices 310, 320, and 100. In a minimal configuration, system 400 can merely include a portable data processing device 100 with a display screen 120, for example a portable computer, tablet computer, smart phone, or other type of portable data processing device, and an image capturing device 110 that is either integrated or operatively attached to portable data processing device 100, for example an external camera device, to provide image data to portable data processing device 100 for performing one or more steps of the method 300.

With step M10 of method 300, the patient or living being L that will be undergoing spinal correction surgery is scanned by medical imaging device 310 so that his spinal column SC, in an uncorrected state pre-surgery, or a part thereof can be viewed as an image, either by a print-out, or can be digitized and displayed on a computer screen, for example by transmitting data on the results of the medical imaging of the uncorrected spinal column SC from medical imaging device 310 to data processing device 320 and displayed on display screen 330. Step M10 can be performed by different types of medical imaging devices 310, for example but not limited to radiology devices, computer tomography (CT), multi-detector CT (MDCT), magnetic resonance imaging (MRI), ultrasound scanning such as but not limited to spinal sonography or ultrasonography, fluoroscopy imaging, surgical X-ray imaging device, for example but not limited to upright serial radiography, image capturing with upright biplanar slot scanners, either with 2D or 3D imaging, as long as they are capable of providing imaging data that includes imaging information on the spinal column SC of the patient or living being L, where individual vertebrae V of spinal columns SC can be identified. In this step, image data captured by medical imaging device 310, including image data of uncorrected spinal column SC can be transferred and further processed by a data processing device 320, the data processing device 320 equipped with a display screen 330, as exemplarily shown in FIG. 1B. Image data of uncorrected spinal columns SC can include but is not limited to one or more radiography images with different orientational views of spinal column SC, for example a back and a side view, can include image slice data of spinal column SC, or can include three-dimensional imaging data of spinal column SC. As indicated above, other data processing device can also be used to receive image data of uncorrected spinal column SC, for example but not limited to portable data processing device 100.

With step M20, image data of the uncorrected spinal column SC1 can be displayed on a display screen 330 that can be operatively associated to data processing device 320, and based on this image data from medical imaging device 310, different parameters and values of the spinal column SC1 can be calculated with computer instructions, for example by using image processing algorithms that allow to detect the different vertebra V of uncorrected spinal column SC1, and to detect a geometric position and orientation of the different vertebra V of uncorrected spinal column SC1, and other parameters, as next discussed. Hereinafter, the uncorrected spinal column is referred to as SC1, while the corrected spinal column is referred to as SC2.

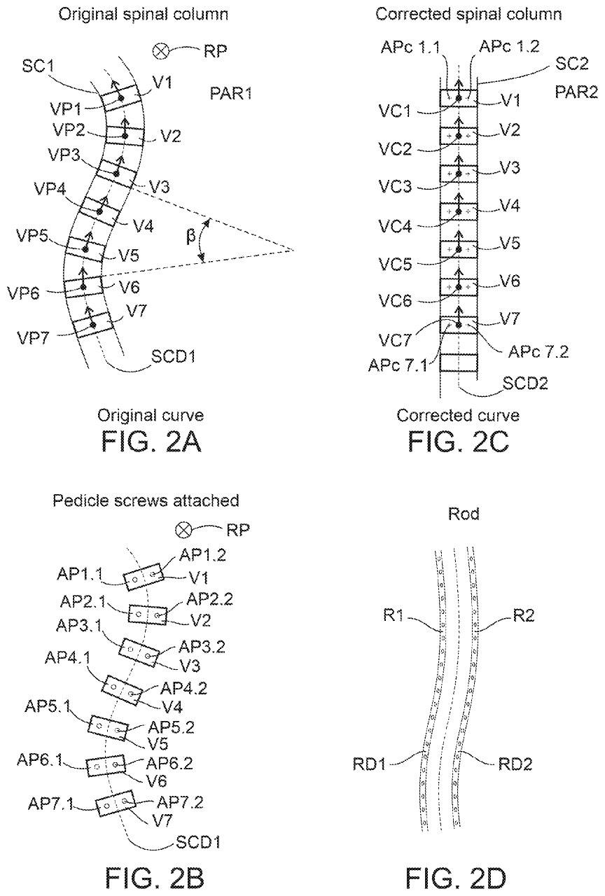

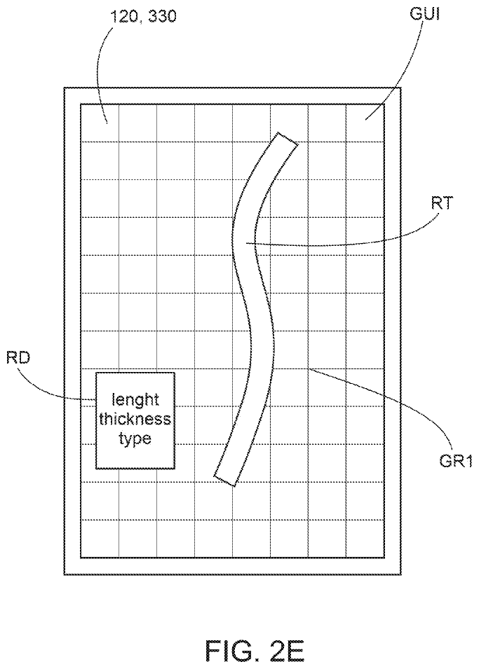

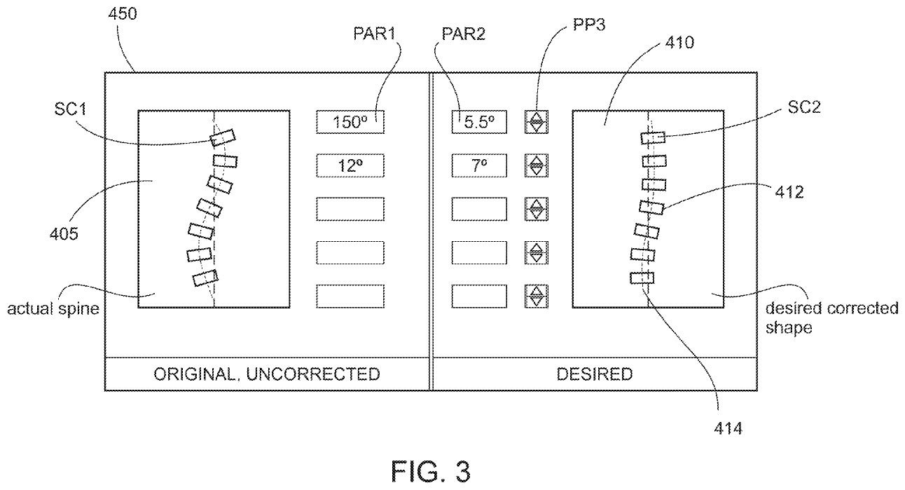

For example, with step M20, data processing device 320 can calculate pose data information PDI_V for each vertebrae V based on the imaging data from step M10, for example to calculate three-dimensional (3D) position and orientation information VP for each vertebra V, for example seven (7) data sets VP1 to VP7 for seven (7) exemplary vertebra V1 to V7, the number seven (7) being merely exemplary, as visualized in FIG. 2A. For example, coordinates and orientation information can be generated, referenced to a three-dimensional cartesian coordinate space, and can be calculated for each vertebrae V1 to V7, for example including a three-dimensional coordinate data for each vertebrae V1 to V7, and an orientation or directional information, for example a vector, for each vertebrae V1 to V7.

The orientation and position data VP for the vertebrae V can be referenced to a chosen or given reference point RP, for example a reference point RP at a position or location given by the placement of the medical imaging device 310, a reference point RP that is provided by an radiopaque marker in the field of view of the imaging area of the medical imaging device 310, thereby visible or detectable in the captured medical images, for example a dynamic reference frame (“DRF”) that is placed on the body of the patient or living being L, a reference point RP based on a bone or other body location of the patient or living being L, for example a location at the hip, one of the vertebrae, or the skull, thereby using a reference point that is innate to the patient or living being L. Preferably, a reference point RP is used that is fixed or otherwise provided to the body of the patient or living being L. A detection of reference point RP and determination of its geometric coordinate location can be done by image data processing as a part of step M20 when performing data processing on the imaging data of step M10, for example by a pattern matching and tracking to detect an optical marker or other pattern that represents the reference point RP, for example with the help of an artificial intelligence network. This can be done a data processor of data processing device 320, or by a data processor of medical imaging device 310, or by another data processing device, for example one that is in operative connection with a cloud or remote server.

While the orientation information for each vertebra V can be simply a direction of orientation of the corresponding vertebra V in space, the location information could be a center of gravity of the vertebra V, for example a volumetric three-dimensional determination of the center of gravity, center of mass, center of rotation of vertebra V, or can also be based on a simplified calculation based on the two-dimensional determination of a centroid or geometric center or center of area if based on two-dimensional image information. Preferably, the coordinate position VP for the location information corresponds to or approximates a center of rotation of the corresponding vertebra V.

In addition, with step M20, data processing device 320 can be configured to determine a spinal curve data SCD1 of the original, uncorrected spinal column SC1. This can be done by using an approximation of a curve that fits the different three-dimensional coordinate positions VP1 to VPn of the different vertebra V1 to Vn that have previously been determined with the pose data information PDI_V for each vertebrae V, for example by interpolation or by using a smoothing curve with a regression analysis. In a variant, spinal curve data SCD1 can be directly calculated from image data of the scans of medical imaging device 310, for example by using a trained neural network or other type of artificial intelligence, to determine parameters of spinal curve data SCD1 directly from image data, for example based on the X-ray images from medical imaging apparatus 310, without first using or determining the pose data information PDI_V.

In addition, with step M20, data processing device 320 can be configured to process image information of the uncorrected spinal column SC1 to determine different parameters that characterize spinal column SC1, herein referred to as different parametrization values PAR1 for the spinal column SC1, these preferably including parameters that characterize the spinal deformity of living being or patient L, for example parameters that describe different types of spinal deformities such as but not limited to Scoliosis, Lordosis, Kyphosis. The parametrization values PAR1 can include but not limited to Coronal Angle Cobb angle, Axial Angle, Sagittal Angle, cervical, thoracic, lumbar parameters, pelvic incidence (PI), pelvic tilt (PT), sacral slope (SS), lumbar lordosis, thoracic kyphosis, sagittal vertical axis, sagittal spinal curvature, Ferguson angle, Greenspan index, TRALL angle, Centroid method. Different computer algorithms can be used to analyze the medical imaging data provided by step M10, to automatically calculate different spinal parametrization values PAR1, for example by using artificial intelligence as shown for example in the following scientific publications: Zhang et al., “Computer-Aided Cobb Measurement Based on Automatic Detection of Vertebral Slopes using Deep Neural Network,” International Journal of Biomedical Imaging 2017, Rajnics et al., “Computer-Assisted Assessment of Spinal Sagittal Plane Radiographs,” Clinical Spine Surgery, Vol. 14, No. 2, year 2001, pp. 135-142, or for example Horng et al., “Cobb Angle Measurement of Spine from X-ray Images Using Convolutional Neural Network,” Computational and Mathematical Methods in Medicine, 2019, Thalengala et al., “Computerized Image Understanding System for Reliable Estimation of Spinal Curvature in Idiopathic Scoliosis,” Scientific Reports, Nature, Vol. 11, No. 1, year 2021, pp. 1-11, Vrtovec et al., “A Review of Methods for Quantitative Evaluation of Spinal Curvature,” European Spine Journal, Vol. 18, No. 5, year 2009, pp. 593-607. These calculations can be based on the three-dimensional coordinates of the uncorrected spinal column SC1 with spinal curve data SCD1 that can be determined as further explained above, for example using a vertical axis as a reference axis, or can also be directly calculated by image processing from the 2D or 3D images from the medical imaging device 310.

However, it is also possible that after the step M10 of imaging the uncorrected spinal column SC1, the parameters of the spinal column SC are manually determined with step M20, for example based on radiography imaging data displayed on display screen 330, for example as discussed in the publication of Malfair et al., “Radiographic Evaluation of Scoliosis,” American Journal of Roentgenology, Vol. 194, No. 3_Supplement, year 2010, pp. S8-S22, in the context of Scoliosis. This data on parametrization of the spinal column SC1 can then be entered to data processing device 320 by the user or operator O, for example with the keyboard or by the use of graphical elements of a graphical user interface.

In addition, with step M20, data processing device 320 can also be configured to perform an identification algorithm that allows to identify which type and number of vertebra V has been detected, for example to determine if it is one of the cervical vertebrae C1 to C7, if it is one of the thoracic vertebrae T1 to T12, if it is one of the lumbar vertebrae L1 to L5 or L6, or if it is one of the sacrum vertebrae SI to S5. This data can be part of the pose data information PDI_V, such that each detected vertebra V is identified as to its type and number and this data is provided to the pose data information PDI_V, and associated with a coordinate and orientation data in space. This part of step M20 can used different types of artificial intelligence and trained networks, for example see for example the following scientific publications: Lecron et al., “Heterogeneous Computing for Vertebra Detection and Segmentation in X-ray Images,” International Journal of Biomedical Imaging, year 2011, Benjelloun et al. “Spine Localization in X-ray Images Using Interest Point Detection,” Journal of Digital Imaging, Vol 22, No. 3, year 2009, pp. 309-318, Lecron et al., “Fully Automatic Vertebra Detection in X-Ray Images based on Multi-Class SVM,” In Medical Imaging 2012: Image Processing, col. 8314, p. 83142D. International Society for Optics and Photonics, year 2012, Ebrahimi et al., “Vertebral Corners Detection on Sagittal X-Rays Based on Shape Modelling, Random Forest Classifiers and Dedicated Visual Features,” Computer Methods in Biomechanics and Biomedical Engineering: Imaging & Visualization, Vol. 7, No. 2, year 2019, pp. 132-144, Dong et al . . . “Automated Vertebra Identification from X-ray Images,” In International Conference Image Analysis and Recognition, pp. 1-9. Springer, Berlin, Heidelberg, 2010, see also All Answers Ltd. Algorithms for pre-processing and processing stages of x-ray images [Internet]. November 2018. [Accessed 16 Jun. 2021]; Available from: https://nursinganswers.net/essays/algorithms-for-pre-processing-and-processing-stages-of-x-ray-images.php?vref=1. This data on vertebra identification can be visualized with a simplified and exemplary representation of the original and uncorrected spinal column SC1 in FIG. 2A, for example together with graphical primitives or other graphical elements to visualize the different vertebra types and numbers.

In this example, seven (7) vertebrae are detected, or selected, and processed to extract pose data information PDI_V, but this number is only exemplary, there could be a smaller or a higher number of vertebrae V, for example n positions VP1 to VPn, n being a number between two (2) and a maximal theoretical number of thirty-three (33), thirty-three being the number of vertebra V of a human being. For spinal correction purposes, the number of selected vertebrae V would not be thirty-three (33), as this number includes five (5) sacral vertebrae, four (4) coccygeal vertebrae, and seven (7) cervical vertebrae, that are not corrected for spine deformation correction purposes, but a smaller number, as corrective spinal rods R are not attached to all vertebrae V of a spinal column SC. For most surgeries, only a certain number of the twelve (12) thoracic vertebrae and a certain number of the five (5) lumbar vertebrae are used. As indicated above, this data PDI_V can also include identification information to identify to what type and number of the vertebra V of the spinal column SC of living being L belongs to.

Step M25 can be performed based on the displaying and processing the imaging data of steps M10 and M20, where data processing device 320 can be configured to provide for a user interface, for example a graphical user interface, that allows user, surgeon, or operator O to manually select different vertebrae V that are displayed on the display screen 330 with an input device, for example with a computer mouse, keyboard, touchpad, touch screen or other type of data input device, to select vertebra V to which the pedicle screws PS will be attached to, as the vertebra V of interest. It is also possible to manually enter identification information to select different vertebrae V of interest, for example via a text editor or command prompt. For this purpose, the vertebrae can be graphically labelled with their type and number with step M20, to facilitate the task. Basically, surgeon or operator O needs to determine which vertebrae V need to be attached to a spinal correction rod R for performing the corrective back surgery, and he or she can make this finding based on the displayed imaging data of spinal columns SC1. Therefore, surgeon or operator O selects a certain number of vertebrae V of interest to which the surgeon or operator O would like to attach a pair of pedicle screws PS thereto, for ultimately attach a spinal correction rod R for spinal deformity correction. The calculation of pose data information PDI_V can be reduced to the selected vertebrae V, as pose data information for the unselected vertebrae that are not of interest is not necessary for the remaining steps of the method 300. Thereby, surgeon or operator O provides for information as to which vertebrae V will be used for the correction by a spinal correction rod R.

The selection of the vertebrae V can be done, for example, by graphically selecting the individual vertebra V on the display screen 330 with a graphical user interface (GUI), for example by generating graphical primitives or other graphical elements to highlight or otherwise show the vertebrae V as a graphical overlay to better illustrate or highlight the vertebrae V on the imaging data, touching or otherwise graphically selectin the different graphical primitives representing the different vertebra V, for example by graphically selecting a section of the spinal column SC1. This step M20 is similar to the step C70 described in International Patent Application No. PCT/IB2021/051694, this reference herewith incorporated by reference in its entirety. See also the scientific publication of Grigorieva et al., “The Construction of an Individualized Spinal 3D Model Based on the X-Ray Recognition,” In 2018 23rd Conference of Open Innovations Association (FRUCT), pp. 143-149. IEEE, year 2018, and Manni et al., “Towards Optical Imaging for Spine Tracking Without Markers in Navigated Spine Surgery,” Sensors, Vol. 20, No. 13, year 2020, p. 3641.

The graphical labelling or highlighting of the different vertebrae V of uncorrected spinal column SC1 can be part of step M20, it is possible to display a medical image from medical imaging device 310 of spinal column SC from the imaging data of step M10, a graphical model of spinal column SC, or an original medical image with a graphical overlay, for example with graphical primitives that represent the individual vertebrae V. For example, step M20 can be performed based on one 2D image of spinal column SC1, for example an X-ray image, or can be based on two or more 2D images of spinal column SC1, for example a back or front X-ray image, and a side view X-ray image. In a variant, it is also possible that 3D images are used for this data processing step, to extract pose data information PDI_V of the original and uncorrected spinal column SC1, for example magnetic resonance or computer tomography images such as but not limited to short-T1 inversion recovery magnetic resonance (STIR-MRI) or three-dimensional CT scanning.

For example, with step M20, it is possible that user, operator, or surgeon O can execute different calculations by the data processing device 320 with respect to the pre-surgery, pre-corrected spinal column SC1. For example, by use of the graphical user interface (GUI), user, operator, or surgeon O can select two different vertebra Vi and Vj, and thereafter can use a software function or module of data processing device 320 to calculate different parameters between these two vertebrae Vi, Vj. For example, as visualized in FIG. 2A, user, operator, or surgeon O could have selected graphically, either by touch screen, computer mouse, keyboard, touchpad, or other type of user input device, vertebra V3 and V6, and thereafter the application software can calculate different parameters between these two vertebra V3 and V6, for example an angle of relative orientation ß therebetween, a distance between these two vertebra V3 and V6 along the spinal curve SCD1, and other parameters, values, or characteristic data of spinal column SC, for example parametrization values PAR1 for the spinal column SC1 enumerated above.

In sum, step M20, in collaboration with step M25, can provide for an user interface for user, surgeon, or operator O to visualize and analyze the uncorrected spinal columns SC1, and can also provide for the tools to calculate and determine different types of data for characterize uncorrected original spinal columns SC1, including pose data information PDI_V of the vertebrae V, data on parametrization values PAR1 of uncorrected spinal column SC1, for example but not limited to the Cobb angle, and data on the curvature of the spinal column SC1, being the spinal curve data SCD1, and allows to display the uncorrected original spinal column SC1 and other data on a display 330 of data processing device 320.

Next, first stages of the orthopedic surgery can be performed with a step M30. In this step, user, surgeon or operator O can make a surgical incision SI into the back of living being or patient L along the spinal column SC, open up the surgical incision SI for access to the vertebrae V for example with a tissue distraction tool, and pairs of pedicle screws PS with screw heads SH for holding spinal rods are attached to the concerned vertebrae V, for example by using a drill or awl, a tapping screw, by the use of a K-wire, pin, or Kirschner wire, and ultimately placing and attaching the pedicle screws PS, or by other surgical methodology for attaching pedicle screws PS. For example, pedicle screws PS can be attached by the use of screw extenders SC, the screw extenders SC pointing out from the surgical incision SI, for example but not limited to the ones discussed in U.S. Pat. No. 10,058,355, this reference herewith incorporated by reference in its entirety. At this stage, screw extenders SC are not removed from the heads of pedicle screws PS. In step M30, preferably, the pedicle screws PS are attached to selected ones of the vertebra V, also referred to as the vertebra of interested selected by user, surgeon or operator O with step M25.

Step M30 can also include the operation of a robot RO, for example a robotic surgery device or a robot device for robot-assisted surgery, for at least a part of the surgical procedure, for example for making the surgical incision SI to the back of the living being or patient L, for opening up the surgical site for placement of the pedicle screws PS, for drilling holes and bone structures to the vertebra V at designated locations for the K-wires and the pedicle screws PS, for screwably or otherwise attaching pedicle screws PS to vertebrae. At least parts of these surgical tasks can be partially or fully performed by a robot surgical device, for example but not limited the use of the da Vinci Surgical System from Intuitive Surgical Inc., Mazor X Stealth Edition Robotic Guidance System by Medtronic, ROSA robot by Medtech, the ExcelsiusGPS robot by Globus Medical, and the SurgiBot and ALF-X Surgical Robotic systems, both from TransEnterix. Based on the information that has been provided by steps M10 and M20, coordinate data of the position of the vertebra V is available as pose data information PDI_V, in addition referenced to a reference frame that can be localized by the robot, can be used. See for example the scientific publications Lieberman et al., “Robotic-Assisted Pedicle Screw Placement During Spine Surgery,” JBJS Essential Surgical Techniques, Vol. 10, No. 2, year 2020, Wang et al., “Robot Assisted Navigated Drilling for Percutaneous Pedicle Screw Placement: a Preliminary Animal Study,” Indian Journal of Orthopaedics, Vol. 49, year 2015, pp. 452-457. In this step M30 where robot surgery can be performed, CT images as medical imaging data from medical imaging device 310 from step M10 can be uploaded to the controller of robot RO, including the data generated from step M20, so that the planning of the placement of the pedicle screws can be done, for example based on a 3D virtual model of uncorrected spinal columns SC1. Next, it is also possible that robot RO can perform a drilling and a screwable attachment of the different pedicle screws.

Step M30 can also include a step of using augmented reality to show the vertebra V of the spinal column as graphical primitives or as a rendered graphical model on a display screen 120 of data processing device 100, while simultaneously filming or providing a life video feed of the back of the living being or patient L with camera 110. This can allow user, operator, or surgeon O to more precisely locate the vertebrae V, and also allows to make the surgical incision SI for the spinal operation at a more precise location. For example, user, operator, or surgeon O can use data processing device 100 having a dedicated application software to film or capture images of around the area of the surgical incision SI of living being or patient L with integrated camera 110, and simultaneously display these images as a live video feed on a graphical user interface on the display screen 120. In addition, the application software can be configured to overlay graphical elements onto the live video feed of the graphical user interface, the graphical elements being a rendered graphical representation of one or more vertebra V, or the entire spinal column SC of the living being or patient L, to create an augmented reality video feed with the live filming of the back of patient or living being L.

With step M30, for properly matching the augmented reality projection of graphical primitives to a live video feed, a reference position between the graphic rendering of the primitives and the real word position of the vertebrae V needs to be used. This can be done by use of a reference frame or marker, for example a dynamic reference frame (“DRF”) that may have been placed on the back of the patient or living being or patient L, for example a plurality of marker points, ruler, or other graphical element that have radiopaque properties for detection from medical images captured by medical imaging device 310 and visibility properties for detection from images captured by camera 110. This allows to make the reference frame visible for both the medical images from medical imaging device 310, based on which the geometric, three-dimensional data of a graphical model of the spinal column SC1 can be based, and also visible to the live video feed, and thereby detectable by an image processing algorithm and used for the augmented reality rendering. For example, the reference frame can be in the form of a longitudinal ruler with different identifiable markers, placed along spinal column SC1 on the skin of patient or living being L, in parallel to spinal column SC1, with both radiopaque and visible properties.

Next, with a data processing device 100 that includes an image capturing device 110, surgical incision SI and the screw extenders SE can be filmed, scanned, or images can be captured, to detect a position of the screw extenders, with a step M40, with the goal to detect locations or geometric positions, where the spinal fixation rod R will be attached to. For example, data processing device 100 or data processing device 320 can instruct an image capturing device 110, for example an internal camera element or an external camera with framegrapper, to capture images from an area of surgical incision SO, the captured images thereby being provided to a data memory of data processing device 110, 320. This can be executed by operator or surgeon O, or an assistant, using data processing device 100, for example a smart phone having the appropriate application software and equipped with a camera or other type of image capturing device 110, activating the application installed on data processing device 100 for filming a location of the surgical incision SI and thereby capturing images of the screw extenders SE, or can be done by activating a fixedly installed camera, or a plurality of cameras that are in operative connection with data processing device. This step can also include the filing, scanning or otherwise capturing image of a reference point RP, as further explained below with respect to step M45. This step M40 includes the steps U30 and C10 of the method 200 of International Patent Application No. PCT/IB2021/051694. This step can be further enhanced by the augmented reality features, where graphical primitives GP are displayed, for example to highlight screw extenders SE or highlight screw heads SH, or both, with step D25 of method 200. This calculation can also be performed by another data processing device, for example data processing device 310, or other electronic device that includes an image-capturing means, for example a digital camera. With this step M40, it is not necessary to use a medical imaging device to determine the position of the pedicle screws PS relative to the vertebra V, but a simple imaging data capturing and processing step can be performed, thereby avoiding the exposure of patient or living being L to additional radiation of a medical imaging device 310. As explained in International Patent Application No. PCT/IB2021/056242, this step C10 can also include the detection of the screw heads SH in the surgical incision SI without them being connected to screw extenders SE, or a combination thereof, for example by detection techniques involving the use of RFID tags attached to screw extenders SE or screw heads SH of pedicle screws PS, optical marker devices removably attached to screw extenders SE or screw heads SH or directly provided on screw extenders SE or screw heads SH, pattern matching for detecting partially covered optical markers or even screw head SH itself, thermal imaging to more easily find screw heads SH inside surrounding tissue and bone of surgical incision SI. In a variant, it is also that this step is performed before the pedicle screws PS are attached to the different vertebrae V of interest, for example by detecting the different K-wires, pins, or Kirschner wires that are attached to the vertebrae, detecting pedicle markers that itself are attached to K-wires or Kirschner wires, or by dedicated optical markers that are placed on the pedicle markers or directly to the K-wires or Kirschner wires, similarly as shown in International patent application No. PCT/IB2022/051805 with steps U230 and C210 of method 600. Based on a detected position of a K-wire, Kirschner wire, pin, pedicle marker, or other specific optical marker, it is possible to calculate or at least estimate a position of a respective attachment point AP for a corresponding pedicle screw PS that will be attached at a location of the K-wire. Thereby, in this variant, step M30 of the attachment of the pedicle screws PS can be performed after the scanning step M40, for example anytime before step M70 is performed, where the rod R or pair of rods R1, R2 need to be attached to the pedicle screws for correction.

Next, also with step M40, user, operator, or surgeon O may be able to select different screw extenders SE that can be taken into account for the calculation of the position and geometry of the spinal column SC, and for proposing spinal rod data RD1, RD2 for a pair of rods R1, R2 by the use of steps D30 where a selection interface is shown to user, operator, surgeon O, and the selection is done by the user input step U40, of method 200. In a variant of method 200, all the detected screw extenders SE can be automatically selected for proposing spinal rod data RD of a corresponding rod R, instead of requesting user feedback for selecting different screw extenders SE, by steps D30 and U40 of International Patent Application No. PCT/IB2021/051694.

In addition, method 300 can include a step M45, that can also be a substep M45 of step M40, of calculating, recalculating, or transforming the geometric positions of the attachment points APn. 1 and APn.2, relative to a reference point RP of the data that characterizes uncorrected spinal column SC1, for example relative to a reference point RP that was detected in step M10 and used by step M20 to calculate and reference spinal curve data SCD1 and to calculate the pose data information PDI_V of the three-dimensional (3D) position and orientation information VP1 to VP7 of the vertebra V of the uncorrected spinal column SC1. For example, step M40 can first use a first reference point for the calculation of the coordinate data of the different attachment points AP, and thereafter, step M45 can be performed where the coordinate data of attachment points AP is recalculated or geometrically transformed to be based on a second, different reference point RP of step M20. In this variant, the geometric positioning of the first and second reference points RP can be known to directly perform the geometric transformation. This can be the case if a first reference point RP is that is visible to the medical imaging data of step M10 of medical imaging, for example a bone, bone part, or radiopaque marker, while a different optically visible marker is used as the second reference point RP for the image capturing of step M40. In a variant, during the filming or scanning of the screw extenders SE, or optionally the pedicle marker, K-wires, optical markers, the reference point RP of step M20 can be optically captured and detected, to serve as a coordinate basis for coordinate data of the different attachment points AP, and thereby no additional geometric transformation to a different reference point RP is needed, and step M45 is thereby not necessary. It is also possible that step M45 includes a step of normalization and calibration, to make sure that spinal curve data SCD1 and the pose data information PDI_V is mapped or transformed the real physical dimensions, for example based on a cartesian coordinate system referenced by metric dimensions or other types of dimensions, and this can be performed by the use of two more reference points RP having a known placement and distance from each other, the use of reference points RP in the form of a predefined reference scale, for example a ruler-type reference marker. Data of the attachment points AP before correction is visualized in FIG. 2B, for this example n being from one (1) to seven (7) pairs of pedicle screws PS that have been attached to the seven (7) different vertebra V1 to V7 in step M30, the vertebrae V1 to V7 shown as graphical primitives, to allow for an attachment of two spinal correction rods R1, R2 to each vertebrae VI to V7, fourteen (14) different attachment points arranged in two columns along spinal column SC, with a first column AP1.1 to AP7.1 and a second columns AP1.2 to AP7.2, for example using a coordinate system referenced to reference point AP2 that is provided as an optical marker. Step M40 can include step C20 of method 200 of International Patent Application No. PCT/IB2021/051694.

With step M45, it can ascertain that the coordinate data of the geometric position of the attachment point pairs APn. 1 and APn.2 is in the same reference frame or referenced to the same reference point AP as the geometric position of the three-dimensional (3D) position and orientation information VP1 to VPn of the corresponding vertebra Vn. This can be done with a geometric transformation of attachment points pairs AP to match the corresponding locations on the reference frame of the position and orientation information VP, or vice versa. In a variant, step M45 can involve the determining of ideal virtual attachment points APV based on the position and orientation information VP1 to VPn of the corresponding vertebra Vn, for example based on a prestored geometric relationship for each corresponding vertebrae Vn, and thereafter, a matching of the detected attachment point pair pairs AP to this locations, by minimizing an overall error of the differences between a position of the virtual ideal attachment points APV and the detected attachment points AP. The matching can also be done by a machine learning algorithm, to transform the coordinate data of the detected attachment points AP to the coordinate reference of the detected position and orientation information VP1 to VPn of the corresponding vertebra Vn, for example by using history data as a training of the machine learning algorithm. Thereby, the geometric relationship between an attachment point pair pairs APn. 1 and APn.2 and the geometric position or location VPn of each vertebra N is mathematically defined.

In a variant, for step M45, to find a matching transformation of the geometric position of the attachment point pairs APn.1 and APn.2 that result from the images of image capturing device 100, relative to the position and orientation information VP1 to VPn of the vertebra V, or relative to the spinal curve data SCD1, that result from the medical images captured by medical imaging device 310 with step M10, or both, one or more reference points can be used, for example a reference point RP embodied as an element that can be captured and identified by both images captured by medical imaging device 310 and images captured by image capturing device 110 of data processing device 100. Thereby, one or more geometric locations can be identified in both the images of medical imaging device 310 and the video feed or images of image capturing device 110, so that the different coordinate data can be matched and mapped to each other. For example, as discussed above, a reference frame can be used as a reference point RP, for example radiopaque marker, rulers, or other symbols RM having a specific symbol for positional detection, for example but not limited to a radiopaque and visible ArUco marker symbol, can be detected and a coordinate position identified that can both be detected by a medical imaging device 310, for example X-ray, and by the images of image capturing device 110. This marker RM can be placed or otherwise attached to the body of the patient or living being L, for example with a temporary adhesive, to prevent the marker RM from being moved during the performance of steps M10, M20, M30, and M40.

In another variant, machine learning and artificial intelligence can be used to create a mapping function between coordinates of attachment point pairs APn. 1 and APn.2 to the respective position and orientation information VP1 to VPn of the corresponding vertebra V, based on historic data of vertebra V and positions of attachment points AP, which in turn are defined by a place, orientation, and insertion depth of a pedicle screw PS to the specific vertebra V. Such training data could be used to train a convolutional neural network that allows to map the different positions of attachment point pairs and position of the corresponding vertebrae V.

In a variant of method 300, the step of scanning M10 with the medical imaging device 310 is not performed, and the different data that characterize the original, uncorrected spinal column SC1 is directly gathered from the attachment points AP that have been detected by step M40 and M45, for example as described in International Patent Application No. PCT/IB2021/051694. Thereby, without using any medical imaging data from a step M10, step M40 also can calculate or estimate pose data information PDI_V of the vertebrae V of uncorrected spinal column SC1, parametrization data PAR1 for uncorrected spinal column SC1, and spinal curve data SCD1 of the uncorrected spinal column SC1, based on data on the coordinates of the attachment points AP.

Next, a step M50 can be performed, where the user, operator, or surgeon O can enter information to computing device 320 that characterizes a desired outcome of the spinal correction surgery, for example by entering data on his or her desired parametrization values PAR2 of the desired corrected spinal column SC2 of patient or living being L that he wants to achieve after the back surgery. For example, as exemplarily shown in FIG. 3, by using a graphical user interface 450 that is for example displayed on display device 120, user, surgeon, o operator O can enter or otherwise define different parametrization values PAR2 of a desired shape of spinal column SC2 that he or she desired to achieve post-surgery, for example by providing the desired Cobb angle, the desired sagittal angle, the desired axial angle, Ferguson angle, Greenspan index, or other parameter of the spinal column SC2 that he wants to achieve with the spinal correction orthopedic surgery. In this step, surgeon or operator O may define his or her desired set of parameters PAR2 based on the currently available values for the parameters PAR1, for example have been or currently being displayed by step M20. For example, the chosen PAR2 may not be spine characterization values that correspond to the ideal, healthy spine shape, but could be an approximation to a healthy spine shape, given the existing spinal deformities or injuries that are currently present. Step M50 can also include the generation and display of a graphical or other representation of the currently present spinal column SC1 parametrization values PAR1 of the patient or living being L, these parameters resulting from step M20. This is shown exemplarily in FIG. 3, on the left side, where a spinal column SC1 pre-surgery is shown on display device 120 in a display area 405 that results from the medical imaging scanning of step M10 and calculated by step M20, or calculated by step M40 without the use of medical imaging, or a graphical representation thereof, for example by using graphical primitives to represent the different vertebrae V and their arrangement or pose. Moreover, graphical user interface 450 also shows different parametrization values PAR1 from spinal column SC1, pre-surgery, that may have been determined by step M20, for example by different algorithms that use image processing and deep learning.

On the right side of graphical user interface 450 of FIG. 3, different text boxes are arranged where user, operator, or surgeon O can enter different desired parametrization values PAR2 for the corrected, desired spinal column SC2, for example by selecting a text box and by entering the numerical values with a keyboard or other type of data input device, these parameters configuring a desired curvature and arrangement of the spinal column SC2 post-surgery. Also, display area 410 can be displayed, showing a graphical representation of such corrected desired spinal column SC2, as a graphical modelling and virtual representation, as the corrective orthopedic surgery has not yet happened. For example, a desired spinal column SC2 can be shown that is graphically represented based on graphical primitives that represent the vertebrae 412, showing a center line 414 to visualize the corrections. Graphical primitives can be simple rectangular blocks, or can be graphical elements that graphically depict a 2D or 3D representation of an actual bone vertebra, or other type of graphical representation, for example as a projection of a geometric model and by graphical rendering as discussed in the scientific publications Długosz, et al., “Realistic Model of Spine Geometry in the Human Skeleton in the Vicon System,” Bio-Algorithms and Med-systems, Vol. 8, No. 1, year 2012 p. 123, and Huynh, et., “Development of a Detailed Human Spine Model with Haptic Interface,” Haptics Rendering and Applications year 2012, p. 194. It is also possible that the data for the desired corrected spinal column SC2 is computer generated, based on an algorithm. For example, depending on the weight, heigh, and deformation of the original, uncorrected spinal column SC1, data for the desired, corrected spinal column can be suggested by the computing device 320, for example desired parametrization values PAR2.

Moreover, graphical elements can be shown on the graphical user interface 450 for modifying the different parametrization values PAR2 that configure the spinal columns SC2, for example but not limited to the desired Cobb angle, the desired sagittal angle, the desired axial angle, Greenspan index, etc. For example, this can be done with arrows PP3 as graphical elements that are configured to increment or decrement a given value of in a text or number box for a corresponding one of the parametrization values PAR2. As a variant, these parameters could be visualized directly in display area 410 of graphical user interface 450, and can be graphically modified by user interaction, for example by graphically displayed lines that represent the orientation of one or more vertebrae, or sections of the spinal column SC. Simultaneously, upon modification of one of the parametrization values PAR2, the graphical display can be updated in real-time, so that user can immediately see the changes with the graphical visualization of spinal column SC2, for example by calculating the updated geometric model and the rendering of desired spinal columns SC2 on a display area 410 upon a change in any parameter of the desired spinal column SC2 with a short time delay. This allows to give immediate visual feedback to user, surgeon, or operator O. Also, for example based on the entered parameters PAR2 for a desired spinal columns SC2, step M50 can thereby calculate other data that characterizes the spinal column SC2, for example to calculate pose data information PDI_V of the vertebrae V of a corrected spinal column SC2, spinal curvature data SCD2 of the desired corrected spinal column SC2, and data on the coordinates of the attachment points AC after correction by a not yet determined spinal rod R.

The method 300 can then proceed to step M60 where data for corrective spinal rod data RD1, RD2, are calculated, the data of RD1, RD2, being such that two actual rods R1, R2, manufactured to have the dimensions and curvature proposed by spinal rod data RD1, RD2 would lead to a corrected spinal columns SC2, if attached to the screw heads of the pedicle screw pairs PS, for example with step M70 as further discussed below. Thereby, with step M60, it is possible to provide the data that is necessary to be able to manufacture or otherwise provide patient-specific rod pairs R1, R2 for addressing a specific spine deformity issue. Step M60 is a calculation step that can use different data to determine corrective spinal rod data RD1, RD2. For example, step M60 can calculate corrective spinal rod data RD1, RD2 based on the current parametrization values PAR1 of the uncorrected original spinal column SC2, based on pose data information PDI_V of the different selected vertebra V, based on data of the positional information of the vertebra VP, based on the spinal curve data SCD1 of the uncorrected spinal column SC1, this data resulting from step M20, based on data related to the desired spinal column SC2 that have been entered or otherwise provided by step M50, including but not limited to corrected spinal curve data SCD2, pose data information PDI_V of the different selected vertebra as corrected vertebrae positions VC, and can also be based on the data of the location of the attachment points AP.

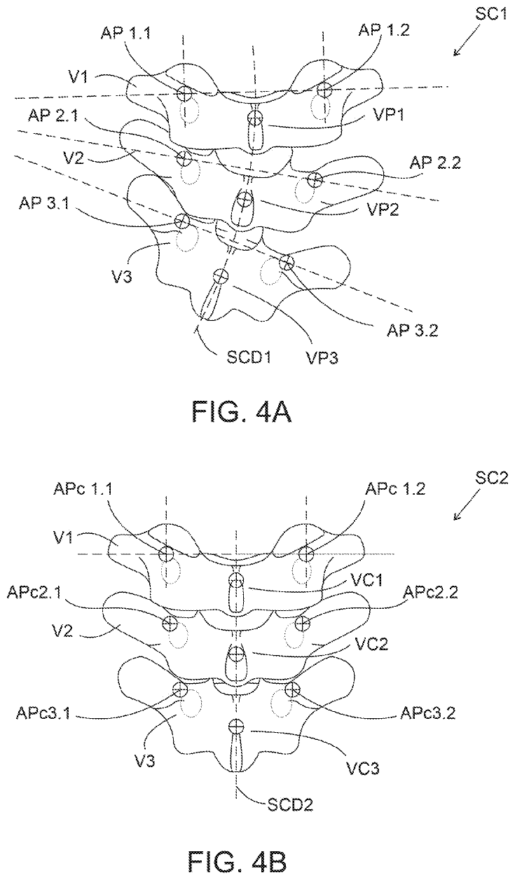

Step M60 can use different types of rod shape analysis and design algorithms, for example by using an Iterative Closest Point-Based Best Fit algorithm to match with the desired locations of the attachment points APc, the corrected attachment points APc illustrated in FIGS. 2C and 4B, for example based on algorithms discussed in the following scientific publications Kokabu et al., “Identification of Optimized Rod Shapes to Guide Anatomical Spinal Reconstruction for Adolescent Thoracic Idiopathic Scoliosis,” Journal of Orthopaedic Research, Vol. 36, No. 12, year 2018, pp. 3219-3224, for example by using the target data and parametrization PAR2 of a desired, corrected spinal column SC2, see for example Solla et al., “Patient-Specific Rods for Surgical Correction of Sagittal Imbalance in Adults,” Clinical Spine Surgery, Vol. 32, No. 2, year 2019, pp. 80-86. Another algorithms can be used, for example finite element based, taking the mechanical stresses applied to the rod R into account, see for example Agarwal et al., “Towards a Validated Patient-Specific Computational Modeling Framework to Identify Failure Regions in Traditional Growing Rods in Patients with Early Onset Scoliosis,” North American Spine Society Journal (NASSJ) Vol. 5, year 2021, p. 100043. The calculation of the new rod data RD1, RD2 can also be knowledge based, for example based on historic data on spinal correction from different back surgery patients, for example by the use of artificial intelligence. Also, it is also possible that the rod calculation of step M60 provides for certain parameters to user, operator, or surgeon O as guidelines to provide for rods R1, R2, indicating a suggested rod type, rod thickness, rod length, and rod shape, rod categories, and that user, operator, or surgeon O uses this information to manufacture a rod with a step M68. This information can be provided on a display screen 330, 120, for example window of the graphical user interface GUI.

In this respect, while the end result of step M68 can be a real rod R that can be placed to be connected to screw heads SH that are accessible via one or more surgical incisions SI for attachment by surgeon, operator or user O with step M70, there can be many different aspects that can be part of step M68 to prepare, suggest, and manually or automatically manufacture a rod R. For example, step M68 can include a step of displaying a rod template RTD to scale (1:1 scale) on the graphical user interface (GUI) of a display device 120, 330, based on rod data RD1, RD2 as exemplarily illustrated in FIG. 2E, as a graphical element showing an outline, shaded line, or a filled line, for example having the line width corresponding to the diameter of the rod R that will be used, or other graphical representation. Also, a graphical representation of a grid GRI can be overlayed over the display area of displayed rod template RTD, for example a matrix of lines interspaced by centimeters or inches. Also, a display window can be provided with different data that characterize the rod R that would result from the displayed rod template RTD, for example overall length along the curved line of rod R, absolute length, maximal bending radius, allowing to provide for some key data that the surgeon, operator or user O, or a technician can use to verify when manufacturing the rod R. For simplification and practical purposes, although a rod R could have a three-dimensionally shaped bending and curving, the displayed rod template RTD could be displayed by one or more two-dimensional views, for example representing a view of the rod R in a direction that is perpendicular to a back of patient or living being L, towards the back, when placed into the pedicle screw heads SH. However, it is also possible to display another side view of rod R with a second displayed rod template RTD. It is also possible that step M68 include a data outputting step to provide rod template RTD to another medium, for example a printer that can print a 1:1 scale of one or more views of the rod R onto a sheet of paper or sterilizable plastic sheet, for example the same view or two views provided onto a GUI of display device 120, 330, or a three-dimensional printer for printing the rod R, for example a 3D titanium printer, for example 3D printer based on titanium powder bed fusion such as laser powder bed fusion or electron beam powder bed fusion, or other titanium 3D printing processes such as direct energy deposition (DED), rapid plasma deposition (RPD), and binder jetting. For simplification and illustration purposes, in the context of step M68 only one rod R is mentioned, but generally two rods R1, R2, can be determined, displayed, printed, and manufactured, for example by displaying two different rod templates RTD1, RTD2, as rods R1, R2 are generally not identical, and depend on the series of attachment positions AP that are form a column on the left side or on the right side of spinal column SC.

With respect to manual manufacturing of the rod R with step M68, based on the 1:1 scale displayed rod template RTD on display 120, 330, or a printed out version on a sheet of paper, surgeon, operator, user O, or a surgical technician can manufacture the rod R to scale, for example by the use of a bendable rod sample that can be cut and bent to the displayed shape, for example directly on display device 120, for example using different types of tools, for example by using a moldable and easy-bendable template rod, to thereafter manufacture a real rod R with a rod replication technique, as shown in International Patent No. WO2020/095262, this reference herewith incorporated by reference in its entirety, or by using other types of rod bender and rod cutting tools. For example, rod R or two rods R1, R2, can be bent and cut by surgeon, operator, or user O directly and intraoperatively from a sterilized straight rod, by the use of rod bending and processing tools, based on information of the rod template provided on display 120, 330. It is also possible that spinal rod data RD1, RD2 is sent to a rod manufacturing facility, for example an external medical device manufacturer, for manufacturing a personalized rod based on the data of RD1, RD2. Rods R can also be manufactured by additive manufacturing methods, for example by three-dimensional printing.

At this stage, based on the step M40 where the screw extenders SE were scanned, detected pre-surgery, and the location of the attachment points AP were determined pre-surgery, as exemplarily shown in FIG. 2B, for calculating a data for a pair of rods R1, R2, for bringing spinal column to the desired corrected position SC1, the method can calculate or estimate at position of the corrected attachment points APc how they are expected they would be post-surgery, based on the data of the desired corrected spinal column SC2, for example the parameters PAR2 that were entered by user or operator O with step M50.

Therefore, within step M60, or a separate step after step M50 of entering parameters PAR2 and scanning step M40, step M55 can be performed, where data on the location of the corrected attachment points APc are calculated, based on the information obtained from step M50 of the desired, corrected spinal columns SC2, for example the PAR2. This is visualized with FIGS. 4A and 4C, showing a back view of three exemplary vertebra VI to V3, with FIG. 4A showing a deformed spine SC1 pre-surgery, and FIG. 4B showing a corrected desired spine SC2, as desired for post-surgery. For each vertebra V, with step M20, pose data information PDI_V is available for each vertebra V, including an orientation of each vertebra V, and a geometric location VP of each vertebra V. For example, step M55 can determine the attachment points APc based on pre-stored data of an ideal spine curvature that is based on data from healthy human beings, but adapted or recalibrated to the currently existing attachment points AP of uncorrected spinal column SC1, for example to take into account and calibrate the data of corrected spinal column SC2 based on a weight, height, body shape, and other characteristics of patient or living being L. This can be done by using data that characterize the uncorrected spine SC1, for example by using the dimensions of the different vertebrae V, for example height, width, diameter, etc., distances between the adjacent vertebrae, that can be part of the parameters PAR1 from step M20, to thereby correct or adjust an ideal curvature of the corrected spine SC2 to the real dimensions and arrangement of original spine SC1.

Based on the data of the new desired corrected spinal curve data SCD2 from step M50, and the consequently change in position and orientation of each vertebra V from pre-surgery location VP to corrected vertebrae positions VC, a new position for the attachment point pairs APcn. 1 and APcn.2 is calculated by step M55, assuming that the movement and correction departed to the spinal column SC did not change or only marginally changed a location of the attachment point pairs AP relative to the corresponding vertebrae V. For example, this is possible when the poly-axiality of the different screw heads SH of the pedicle screws PS is locked. In this respect, step M55 can include a step of geometrically transforming the set of pairs of attachment points pre-surgery APn. 1 and APn.2, as shown in FIG. 4A, to corresponding desired, new pairs of attachment points post-surgery APcn. 1 and APcn.2, such that all new desired corrected vertebrae positions VCn lie along the new desired corrected spinal curve data SCD2, as shown exemplarily in FIG. 4B. In FIG. 4B, to show an example, spinal curve data SCD2 is shown to be represented by a straight vertical line. To transform the detected positions VPn of each vertebrae V from step M20 to the new desired corrected positions VCn, it can be assumed that the distance between adjacent vertebrae V does not vary, as the spinal column SC of patient or living being L is not or only marginally compressed or stretched, or that a length of a curvature of the section of original and corrected spinal curve data SCD1 and SCD2 between adjacent positions VPn and VPn+1 or VPn-1 does not change. With these geometric transformations, the positional data for pairs of corrected attachment points APcn. 1 and APcn.2 can be calculated, solely based on data determined by step M20, and the desired spine correction data of step M50.

Next, with step M60, based on the positional data for pairs of corrected attachment points APcn. 1 and APcn.2 for the vertebra V that have been selected, or all the vertebra V that have a pair of pedicle screws PS attached thereto, two spinal rod data sets RD1, RD2 can be calculated, for example as a series of discrete locations, or as a geometric function or curve, such that two rods R1, R2, as exemplary shown in FIG. 2D, can be placed into the U-shaped grooves of the screw heads of the pedicle screws PS. Rod data sets RD1, RD2 can also include a length of the proposed rods R1, R2. This calculation can take into account a maximal bending radius or curvature of the material that forms the rod R1, R2, for example cylindrical stainless steel or titanium. The proposed rod data set RD1, RD2 can be used to produce rods R1, R2 that have an ideal shape for correcting spinal column SC to a desired curvature SCD2.

Next, with step M68, rod data set RD1, RD2 can be transformed into manufacturing data for spinal rods, for example as computer-aided design (CAD) data, and a pair of rods R1, R2 can be manufactured to have the curvature and length as described in rod data sets RD1, RD2, with a step M68 of calculating manufacturing data and manufacturing the rods R1, R2. This can be a manual, semi-automated, or fully automated step of fabricating the rods R1, R2, for example by using a rod bending machine or device and rod cutting apparatus.

With step M70, the surgical part of the method can continue performed by user, operator or surgeon O, where the two rods R1, R2 that have been manufactured can be placed into U-shaped grooves of screw heads SH of the pairs of pedicle screws PS that are attached to the vertebra V, for example by using the rod reduction feature and the slit-shaped openings of the screw extenders SE, and set screws that are tightened to the screw heads SH for holding rod R inside screw heads SH. In this step, operator, surgeon, or user O can rearrange the spinal column SC of patient or living being L, to rearrange the vertebrae V so that rods R1, R2 will fit into the screw heads SH. This will allow operator, surgeon, or user O to rearranged spinal column SC to be close to or approximate the desired new corrected spinal curve data SCD2 that was determined in step M50.

Method 300 can continue with a step M80 where the positions of the screw extenders SE that are still attached to the screw heads SH can be scanned and detected, this step being a similar step as step M40, but this time with the rods R1, R2 attached to the pedicle screws PS. This step can be performed by using data processing device 100 and the camera 110, and therefore allows to verify the position of spinal columns SC3 after the rods have been attached thereto, without the need of a medical imaging step that would expose the patient or living being L to radiation. With this step, the real attachment positions APR can be detected, based on pose information of the screw extenders SE, and thereafter, based on the known geometric relationship between the attachment positions AP, and the real position or location VR of vertebrae V, the other parameters and data of corrected spinal column SC3 can be calculated for example the currently present parametrization values PAR3 for corrected spinal column SC3, for example but not limited to the Cobb angle, data on the corrected spinal curve data SCD3, corrected position/location and orientation information VR.

In an ideal case, the data characterizing the desired arrangement of spinal column SC2 and the data characterizing actual corrected spinal column SC3 would be the same or a close match. However, for operator, surgeon, or user O to verify the results of the surgery before closing the surgical incision SI, a step M90 can be performed where the data from steps M50 and M80 can be compared, for example by a step M100 of displaying values or curves of desired and corrected spinal curve data SCD2, SCD3 on the display screen 120, 330, for example display device 330 of data processing device 320, or on the display device 120 of the portable data processor 100. This can be done a graphical user interface and a graphical representation of the desired spinal curve SC2 and the actually achieved corrected spinal curve SC3, by displaying the different parametrization values PAR2 of the desired spinal column SC2 and the parametrization values PAR3 of the corrected or post-correction spinal column SC3, similarly as shown in FIG. 3 but this timing using PAR2, PAR3. Also, it is also possible to display the original, uncorrected spinal curve SC1, and the different parametrization values PAR1 of the original, uncorrected spinal curve for comparison, to show three different views of the spinal columns for comparison. Graphical primitives or a graphical modelling and rendering can be used to display a computer-generated spinal representation for the display with the graphical user interface.

After step M90 of comparing, or after step M100 of displaying, or both, it is possible to perform a reiteration of the steps M60 of calculating a corrective rod with rod data RD1, RD2, step M68 of manufacturing a new rod R, preferably a pair of rods R1, R2, step M70 of placing and attaching the new rods R1, R2 to the pedicle screws PS, and the step f performing step M80 a scan to detect the new position of pedicle screws PS and thereby the vertebrae V, and thereafter a new step M90 of comparing, a new step M100 of displaying. In such loop, first, the currently placed and attached rods R1, R2 can be removed from the pedicle screws PS with a step M95. For example, it may be possible that with the scanning step M80, and calculation of the actual corrected parameters that characterize the spine PAR3, the user or operator O realizes that he or she is not satisfied with the results, for example after comparing PAR2 with PAR3, or by a simple visual inspection of the area of surgical incision SI. For example, it is possible that the rod pair R1, R2 calculated by step M60 and manufactured by step M68 did not depart the desired correction to the spine SC1. Therefore, step M90 of comparing, or step M100 of displaying, or both, can include a graphical element or other data input device that allows user or operator O to go back to perform step M50, to enter a new set of parameters PAR2 for the desired shape and arrangement of spinal column SC1. It is also possible that the method 300 can go back to step M40, where the originally-entered parameters PAR2 are preserved and not re-entered with step M50, but surgical incision SI of patient or living being L is scanned again, after rods R1, R2 have been removed, to redetermine the location of the attachment points AP. For example, after a first attempt of attaching the rod pairs R1, R2 with step M70, the position, arrangement, and orientation of spine SC1 may have moved to new positions, or even during the handling of the patient or living being L during surgery, after attachment of pedicle screws PS with step M30, the vertebrae V may have been moved, leading to an insufficient correction of the spinal column SC3, therefore requiring a new determination of the location of the attachment points APc with step M40, M55, without using new values for PAR2.

With the herein presented aspects of method 300 and system 400, it is possible to reduce the number of medical imaging that needs to be performed, to reduce costs and invasiveness of a corrective back surgery, thereby exposing a body of a patient to less radiation, and avoiding the costs and time of a radiation imaging of a patient or living being L. Medical imaging can be substantially replaced by classical imaging and video capture, by using image processing algorithms to determine different position and locations that characterize the spinal column. As explained above, it is even possible that no medical imaging is performed, or the medical imaging is merely performed as an auxiliary step, to rely on the video capture and data processing of steps M40, M80, based on images taken at the surgical incision SI, to determine a spinal correction. In addition, it is possible to directly propose and manufacture corrective spinal rods R1, R2, right at the place of surgery, without the need of external manufacturing and spinal rod design steps.

While the invention has been disclosed with reference to certain preferred embodiments, numerous modifications, alterations, and changes to the described embodiments are possible without departing from the sphere and scope of the invention, as defined in the appended claims and their equivalents thereof. Accordingly, it is intended that the invention not be limited to the described embodiments, but that it have the full scope defined by the language of the following claims.

1. A method for determining a spinal rod for correcting a curvature of a spinal column of a living being with pedicle screws, comprising the steps of:
detecting a rod attachment position for each pedicle screw by capturing image data from the pedicle screws attached to vertebrae of the spinal column at a surgical incision;
determining first parameters of the uncorrected spinal column with a data processing device;
entering second parameters of a desired arrangement of a desired corrected spinal column; and
calculating data characterizing a corrective spinal rod for achieving the desired corrected spinal column when the corrective spinal rod is attached to the pedicle screws, the data calculated based on the rod attachment positions and the second parameters.

2. The method according to claim 1, further comprising the step of:
performing medical imaging to capture medical imaging data of the uncorrected spinal column,
wherein the step of determining the first parameters calculates the first parameters based on the captured medical imaging data.

3. The method according to claim 1, wherein the step of determining the first parameters calculates the first parameters based on the rod attachment positions of the pedicle screws of the step of detecting, or based data from a step of detecting a position of the pedicle screws by capturing image data from the pedicle screws.

4. The method according to claim 1, wherein the step of detecting further comprises:
detecting the rod attachment position by detecting a position of a screw extender that is attached to the respective pedicle screw, by detecting a position of an optical marker, by detecting a position of a pedicle marker, or by detecting a position of a K-wire, and by calculating the rod attachment position from the position of the screw extender, form the position of the optical marker, from the position of the pedicle marker, or from the position of the K-wire.

5. The method according to claim 1, further comprising the steps of:
manufacturing the corrective spinal rod based on the data of the step of the calculating data;
scanning and detecting the screw extenders that are attached to respective pedicle screws, the pedicle screws in turn fastened to vertebra of a corrected spinal column after the corrective spinal rod is attached to the pedicle screws, and determining a rod attachment position for each pedicle screw;
determining third parameters of the corrected spinal column; and
displaying second parameters of the desired corrected spinal column and the third parameters of the corrected spinal column on a display device.

6. The method according to claim 1, wherein the first parameters include positional and/or orientation information of the vertebra of the spinal column, and parametrization information of the spinal column before departing a correction.

7. The method according to claim 6, wherein the parametrization information includes at least one of a Cobb angle, Sagittal angle, Coronal angle, Axial angle.

8. The method according to claim 1, further comprising a steps of:
matching the rod attachment positions determined by the step of scanning and detecting with the first parameters of the step of determining.

9. The method according to claim 1, further comprising a steps of:
transforming the rod attachment positions of the uncorrected spinal column to new rod attachment positions of the desired corrected spinal column based on the first parameters and the second parameters.

10. A non-transitory computer readable medium having computer code recorded thereon, the computer code configured to perform a method for determining a spinal rod for correcting a curvature of a spinal column of a living being when executed on a data processing device of a computer device, the method comprising the steps of:
detecting a rod attachment position for each pedicle screw by capturing image data from the pedicle screws at a surgical incision;
determining first parameters of the uncorrected spinal column with the data processing device;
entering second parameters of a desired arrangement of a desired corrected spinal column with a data input device that is in operative connection with the computer device; and
calculating data characterizing a corrective spinal rod for achieving the desired corrected spinal column when the corrective spinal rod is attached to the pedicle screws, the data calculated based on the rod attachment positions and the second parameters.

11. The non-transitory computer-readable medium of claim 10, wherein the further comprises the step of:
performing medical imaging to capture medical imaging data of the uncorrected spinal column,
wherein the step of determining the first parameters calculates the first parameters based on the captured medical imaging data.

12. The non-transitory computer-readable medium of claim 10, wherein the step of determining the first parameters calculates the first parameters based on the rod attachment positions of the pedicle screws of the step of detecting, or based data from a step of detecting a position of the pedicle screws by capturing image data from the pedicle screws.

13. The non-transitory computer-readable medium of claim 10, wherein the step of detecting further comprises:
detecting the rod attachment position by detecting a position of a screw extender that is attached to the respective pedicle screw or by detecting a position of an optical marker attached to a K-wire, and by calculating the rod attachment position from the position of the screw extender or the position of the optical marker.

14. The non-transitory computer-readable medium of claim 10, the method further comprising the steps of:
manufacturing the corrective spinal rod based on the data of the step of the calculating data;
scanning and detecting the screw extenders that are attached to respective pedicle screws, the pedicle screws in turn fastened to vertebra of a corrected spinal column after the corrective spinal rod is attached to the pedicle screws, and determining a rod attachment position for each pedicle screw;
determining third parameters of the corrected spinal column; and
displaying second parameters of the desired corrected spinal column and the third parameters of the corrected spinal column on a display device.

15. A computer system for determining a spinal rod for correcting a curvature of a spinal column of a living being, the system comprising:
a data processing device having a data input device;
an image capturing device in operative connection with the data processing device,
wherein the data processing device is configured to
instruct a capturing of image data with the image capturing device from pedicle screws that are attached to vertebrae via a surgical incision of the living being,
detect a rod attachment position for each pedicle screw from the captured image data,
determine first parameters of the uncorrected spinal column with the data processing device,
enter second parameters of a desired arrangement of a desired corrected spinal column with the data input device to the data processing device, and
calculate data characterizing a corrective spinal rod for achieving the desired corrected spinal column when the corrective spinal rod is attached to the pedicle screws, the data calculated based on the rod attachment positions and the second parameters.

16. The computer system of claim 15, further comprising:
a medical imaging device in operative connection with the data processing device, configured to perform medical imaging of the living being to capture medical imaging data of the uncorrected spinal column,
wherein in the determining of the first parameters, the data processing device calculates the first parameters based on the captured medical imaging data.

17. The computer system of claim 15, wherein in the determining of the first parameters, the data processing device calculates the first parameters based on the rod attachment positions of the pedicle screws of the detecting, or based data from a detecting of a position of the pedicle screws by capturing image data from the pedicle screws with the image capturing device.
